(19) United States
(12) Patent Application Publication
(10) Pub. No.: US 2019/0256821 A1
(43) Pub. Date: Aug. 22, 2019

(54) Compositions and Methods for Muscle Progenitor Cell-Based Therapies

(71) Applicant: SANOFI, Paris (FR)

(72) Inventors: Paul August, Oro Valley, AZ (US); Christopher M. Penton, Oro Valley, AZ (US)

(73) Assignee: SANOFI, Paris (FR)

(21) Appl. No.: 16/320,193

(22) PCT Filed: Jul. 27, 2017
(86) PCT No.: PCT/US2017/044170

§ 371 Date: Jan. 24, 2019

Related U.S. Application Data
(60) Provisional application No. 62/367,812, filed on Jul. 28, 2016.

(30) Foreign Application Priority Data
Jun. 16, 2017 (EP) 17305745.6

(51) Int. Cl. C12N 5/077; A61K 35/34

(52) U.S. Cl. CPC C12N 5/0659; C12N 2533/52; A61K 35/34

(57) ABSTRACT

Disclosed herein are methods and compositions that provide for improved production and efficacy of cell-based therapies. For example, the culture of muscle progenitor cells (satellite cells) on laminin 521 is provided as a means to maintain differentiation and engraftment potential of the cells, e.g. for therapeutic purposes.

### FIELD

The present described inventions relate, inter alia, to methods and compositions that provide for improved production and efficacy of cells for use, e.g. in cell-based therapies.

### BACKGROUND

Cell-based therapies provide for exciting possibilities for treatment of various diseases. However, the use of cellular therapy methods is often hindered by inefficient production techniques that can compromise efficacy.

For example, satellite cells are the major effector cell responsible for eliciting muscle regeneration and have potential for use in cell-based treatment of neuromuscular dystrophy diseases. Such treatment requires injection of expanded satellite cells that engraft and incorporate into skeletal muscle fibers. Preparation of these cells is cumbersome as, among others, in vitro mimicking of the satellite cell “niche”—including extracellular matrix (ECM) adhesion proteins which influence satellite cell activity—is often required. Extended passaging of muscle satellite cells using traditional methods and ECMs results in the progressive loss of engraftment potential. Traditional methods lead to poor cellular survival and only minimal integration of injected cells into skeletal muscle fibers.

There is a need for improved compositions and methods for the handling and production of cell-based therapies.

### SUMMARY

Accordingly, in general, methods and compositions which improve the production and utility of cell-based therapies are disclosed herein.

In one aspect, the present invention relates to compositions and methods for producing a progenitor cell, such as a muscle progenitor cell (satellite cell) in which cells are cultured in the presence of one or more laminin α5 proteins, such as laminin 521. In various embodiments, the present compositions and methods produce cells which have a differentiation and engraftment potential that is suitable for use as a cell-based therapy in humans or in drug discovery. In some embodiments, the present compositions and methods comprising one or more laminin α5, such as laminin 521, provide improved cell differentiation and engraftment potential as compared to cells not cultured in laminin α5, such as when cultured in any one or more of laminin 211, laminin 111, fibronectin, gelatin, collagen, hydrogel and matrigel (sometimes referred to as MG) i.e. a gelatinous protein mixture secreted by Engelbreth-Holm-Swarm mouse sarcoma cells, Corning Life Sciences). In some embodiments, the present compositions and methods allow for large-scale expansion and/or long term (e.g. multiple passage in vitro) culture of cells as compared to cells not cultured in laminin α5, such as when cultured in any one or more of laminin 211, laminin 111, fibronectin, gelatin, collagen, hydrogel, and matrigel.

In some embodiments, the present compositions and methods provide increased cellular proliferation during expansion of the cells, for example, in the early stages of expansion as compared to cells not cultured in laminin α5, such as when cultured in any one or more of laminin 211, laminin 111, fibronectin, gelatin, collagen, hydrogel, and matrigel.

In other embodiments, the present compositions and methods provide improved fusion, including following multiple cell passages as compared to cells not cultured in laminin α5, such as when cultured in any one or more of laminin 211, laminin 111, fibronectin, gelatin, collagen, hydrogel, and matrigel.

In some embodiments, the present compositions and methods provide improved fusion and therefore an increase in multinucleated myotubes as compared to cells not cultured in laminin a5, such as when cultured in any one or more of laminin 211, laminin 111, fibronectin, gelatin, collagen, hydrogel, and matrigel. In further embodiments, the present compositions and methods provide increased functional muscle fibers, e.g. when provided to a subject.

In various embodiments, the present compositions and methods provide improved differentiation and diminished spontaneous differentiation as compared to cells not cultured in laminin α5, such as when cultured in any one or more of laminin 211, laminin 111, fibronectin, gelatin, collagen, hydrogel, and matrigel. For example, the present compositions and methods provide for cells that express and/or up-regulate myosin heavy chain (MHC) or comparable differentiation-specific markers, such as, for example, alpha-actinin and troponin-T.

In some aspects, the present compositions and methods provide a cell, e.g. a progenitor cell, such as a muscle progenitor cell (satellite cell), culture on one or more laminin α5, such as laminin 521, which allows for expansion of satellite cells in vitro while maintaining their ability to be used for cell-based therapy applications. For example, the present invention provides methods of treatment of various neuromuscular diseases or disorders in which muscle progenitor cells (satellite cells) are prepared as described herein and implanted into a patient. Such methods find use in the treatment of a variety of neuromuscular diseases or disorders, such as, for example, muscular dystrophies.

### DESCRIPTION OF THE DRAWINGS

FIG. 1 shows a general laminin structure. Laminin 111 is composed of α1, β1, and λ1 chains while laminin 521 is composed of α5, β2, and λ1 chains.

FIG. 2 shows results of a FACS sorting study of freshly isolated mouse satellite cells.

FIGS. 3A, 3B, 3C, and 3D show results of short term cell growth/plating experiments comparing laminin 111, laminin 211, laminin 332, laminin 411, laminin 421, laminin 511, laminin 521, fibronectin (FN), gelatin, and growth factor reduced matrigel (MG). FIGS. 3A and 3C increased proliferation in DBA satellite cells, FIGS. 3A and 3B show increased differentiation in BL6 satellite cells, FIG. 3C shows improved differentiation in BL6 satellite cells, and FIG. 3D shows improved differentiation in BL10 satellite cells.

FIGS. 4A, 4B, 4C, 4D, 4E, and 4F show results of long term cell growth/plating experiments comparing laminin 111, laminin 211, laminin 511, fibronectin (FN), and matrigel (MG). “Lam 1” is laminin 111, “LAM 2” is laminin 211, “Lam 5” is laminin 521, “FN” is fibronectin, and “MG” is matrigel. FIGS. 4A and 4D show extensive myogenic differentiation of laminin 521. FIG. 4B shows superior results in cells that contain two or more nuclei. In FIG. 4C, the histograms are, in each series, from left to right: Lam 5, MG, and Lam 1. FIG. 4C shows that cells on laminin 521 form more multinucleated myotubes than the other substrates. In FIGS. 4E and 4F, the histograms are, in each series, from left to right: Lam 1, Lam 5, and MG. FIGS. 4E and 4F show increased proportions of nuclei per myotube at passage 6 and passage 8, respectively.

FIGS. 5A and 5B show results of substrate transfer experiments. “Lam 1” is laminin 111, “LAM 2” is laminin 211, “Lam 5” is laminin 521, “FN” is fibronectin, and “MG” is matrigel. For FIG. 5B, the order of histograms in each series of expansion substrates is Lam 1, Lam 2, Lam 5, FN, and MG.

FIGS. 6A, 6B, and 6C show results of FACS staining of cells. FIG. 6A shows FACS staining of cells for integrin α7, PDGFRα, or CD31, though there were no detectible PDGFRα and CD31 positive cells present. In FIG. 6A “111” is laminin 111, “211” is laminin 211, “521” is laminin 521, “FN” is fibronectin, and “MG” is MATRIGEL. FIGS. 6B and 6C show FACS staining of cells for Pax7 and MyoD. In FIGS. 6B and 6C “Lam 1” is laminin 111, “LAM 2” is laminin 211, “Lam 5” is laminin 521, “FN” is fibronectin, and “MG” is matrigel and the order of histograms in each series is Lam 1, Lam 2, Lam 5, FN, and MG.

FIGS. 7A and 7B show results of integrin expression studies on different ECMs. FIG. 7A shows the percent of cells positive for each integrin for each ECM. FIG. 7B shows the mean intensity for each integrin for each ECM. The histograms in each series are, from left to right: laminin 111, laminin 211, laminin 521, FN, and MG.

FIGS. 8A and 8B show results of culture experiments on human muscle cells. “Lam 111” is laminin 111, “Lam 211” is laminin 211, “Lam 521” is laminin 521, “FN” is fibronectin, and “MG” is matrigel. FIG. 8A shows MACS/FACS staining of cells. FIG. 8B shows increased growth of cells on laminin 521 over other ECMs within the first week.

FIGS. 9A, 9B, and 9C show results of long term culture experiments on human muscle cells. FIGS. 9A shows the highest amount of differentiation on laminin 521 and MG. MHC expression was assessed in FIGS. 9B and 9C.

FIG. 10 shows human satellite cells expanded at a faster rate on Laminin 521 compared to other substrates following 5 passages.

FIG. 11 shows results of culture experiments on freshly isolated mdx/BL10 cells.

FIG. 12 shows imaging in mice on Day 1 post-injection of satellite cells passaged on different substrates. “Lam 1” is laminin 111, “Lam 5” is laminin 521, and “MG” is matrigel.

FIG. 13 shows imaging in mice on Day 28 post-injection of satellite cells passaged on different substrates. “Lam 1” is laminin 111, “Lam 5” is laminin 521, and “MG” is matrigel.

FIG. 14 shows imaging in mice on Day 1 post-injection of satellite cells passaged on different substrates. “Lam 1” is laminin 111, “Lam 5” is laminin 521, and “MG” is matrigel.

FIG. 15 shows imaging in mice on Day 49 post-injection of satellite cells passaged on different substrates. “Lam 1” is laminin 111, “Lam 5” is laminin 521, and “MG” is matrigel.

FIG. 16 shows staining of satellite cells cultured for 15 passages on laminin 521.

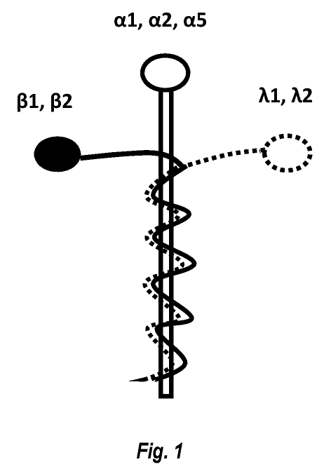

### DETAILED DESCRIPTION

The present invention is based, in part, on the surprising discovery that culture of muscle progenitor cells (satellite cells), including long-term culture (e.g. with multiple passages), with a laminin α5, such as laminin 521, is useful to maintain differentiation and engraftment potential of the cells, e.g. for therapeutic and drug discovery purposes.

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

As used herein, the term “progenitor cell” refers to primary cells or cell lines that are committed to differentiate into a specific type of cell or to form a specific type of tissue (e.g., a muscle progenitor cell).

As used herein, the term “muscle progenitor cell” (used herein interchangeably with “satellite cell”) refers to progenitor cells that differentiate into muscle cells. For example, in mice, the muscle progenitor is integrin alpha 7 positive, and in human, the muscle progenitor is CD56 positive. In some embodiments, the human and mouse muscle progenitor is pax7 positive and/or myoD positive. In some embodiments, the muscle progenitor cell is derived from muscle tissue, and is not a pericyte or a mesoangioblast.

As used herein, the term “laminin α5” refers to extracellular matrix molecules or active fragments thereof encoded by the LAMAS gene (e.g., homo sapiens laminin subunit alpha 5 (LAMAS) mRNA, NCBI Ref. Seq. NM_05560.4, 11445 bp). In some embodiments, the laminin α5 protein is complexed with a laminin β and γ chain. In some embodiments, the laminin β and γ chain can be selected from laminin subunit 1, 2, or 3. In certain embodiments, the laminin α5 may be recombinant or non-recombinant. Examples of laminin α5 include, but are not limited to, laminin 521 (α5β2γ1 chain composition), laminin 511 (α5β1γ1 chain composition), laminin 522 (α5β2γ2 chain composition), laminin 523 (α5β2γ3 chain composition), and active fragments thereof. See also, e.g., 51. Spenlé et al., Cell Adh. Migr. 2013; 7(1):90-100, Macdonald et al., J Struct. Biol. 2010; 170(2):398-405, and Siler et al., Br. J. Haematol. 2002; 119:212-220.

As used herein, the terms “cultured” or “cell cultured” refers to growing cells outside of their natural environment. A “culture” refers to the cells and the structure holding them.

As used herein, the terms “passage” or “passaged” or “subculturing” refers to the process of transferring some cells from a previous culture to a new culture. In some embodiments, one or more passages are conducted. For example, in some embodiments, greater than about 5, 6, 7, 8, 9, 10, 15, or 25 passages are conducted without loss of beneficial properties.

As used herein, the term “fusion capacity” refers to the time it takes to obtain cells with more than one nucleus, and the extent of fusion as a function of the number of myotubes per total number of nuclei and number of nuclei per myotube. “Improved fusion capacity” results in an increase in multinucleated myotubes.

As used herein, “spontaneous differentiation” refers to a cell differentiating without induction.

Cell Production

In one aspect, the present invention relates to compositions and methods for producing a progenitor cell, such as a muscle progenitor cell (satellite cell), in which cells are cultured in the presence laminin α5. For example, in some embodiments, progenitor cells are cultured in a cell medium as known in the art and laminin α5 as the ECM substrate.

The progenitor cells may be primary cells or cell lines. Further, the methods of the invention can be used in vivo, ex vivo, or in vitro. For example, the primary cell can be autologous (derived from and provided to the same subject) or allogenic (derived from and provided to a different subject).

In various embodiments, the present compositions and methods provide cells which have a differentiation and engraftment potential that is suitable for use as a cell-based therapy or in neuromuscular drug discovery.

In some embodiments, the present compositions and methods comprising laminin α5, such as laminin 521, provide improved cell differentiation and engraftment potential as compared to cells not cultured in laminin α5, such as compared to compositions and methods comprising one or more of laminin 211, laminin 111, fibronectin, gelatin, collagen, hydrogel, and matrigel.

In some embodiments, the present compositions and methods provide increased cellular proliferation during expansion of the cells, for example, in the early stages of expansion. In some embodiments, the present compositions and methods provide improved fusion, including following multiple cell passages. In some embodiments, the present compositions and methods provide improved fusion and therefore an increase in multinucleated myotubes. In further embodiments, the present compositions and methods provide increased functional muscle fibers, e.g. when provided to a subject. In further embodiments, the present compositions and methods provide improved differentiation and diminished spontaneous differentiation. In some embodiments, the present compositions and methods provide for cells that up-regulate myosin heavy chain (MHC) or comparable differentiation-specific markers. In some embodiments, any of the features described in this paragraph are increased, decreased or otherwise improved as compared to cells not cultured in laminin α5, such as compared to compositions and methods comprising one or more of laminin 211, laminin 111, fibronectin, gelatin, collagen, hydrogel, and matrigel.

In some embodiments, the present compositions and methods allow progenitor cells to maintain their stem cell and differentiation potential after long term culture.

In some embodiments, the present compositions and methods allow for long term growth without loss of beneficial properties (e.g. for use as a cell-based therapy). For example, in some embodiments, the present compositions and methods support greater than about 5, or 6, or 7, or 8, or 9, or 10, or 15, or 25 passages without loss of beneficial properties (e.g. for use as a cell-based therapy). By way of further example, in some embodiments, the present compositions and methods support greater than about 2500-, or 3000-, or 3500-, or 4000-, or 4500-, or 5000-, or 5500-, or 6000-, or 6500-, or 7000-, or 7500-, or 8000-, or 8500-, or 9000-, or 9500-, or 10,000-, or 11,000-, or 12,000-, or 13,000-, or 14,000-, or 15,000-, or 16,000-, or 17,000-, or 18,000-, or 19,000-, or 20,000-, or 25,000-, or 30,000-, or 35,000-, or 40,000-, or 45,000-, or 50,000-, or 55,000-, or 60,000-, or 65,000- or 70,000-, or 75,000-, or 80,000-, or 85,000-, or 90,000-, or 95,000-, or 100,000-, or 105,000-fold expansion of cells without loss of beneficial properties (e.g. for use as a cell-based therapy).

In various embodiments, the laminin α5 is one or more of laminin 521, laminin 511, laminin 522, laminin 523, or active fragment thereof. In various embodiments, the laminin α5 is recombinant. In various embodiments, the laminin α5 is laminin 521 or active fragment thereof.

In various embodiments, and without wishing to be bound by theory, the laminin α5, e.g. laminin 521, interacts with one or more of with six integrin binding sites (α3β1 (twice), αVβ3, α6β1, α6β4, α7β1). This is distinguishable from the four binding sites in laminin 111 and MG (α1β1, α2β1, α6β1, α7β1).

Combination Culture Agents

In various embodiments, in addition to laminin α5, cells can also optionally be contacted with compounds that promote cell adhesion, proliferation, differentiation, and/or maintenance, including but not limited to any of the collagens, other laminin types, fibronectin, integrins, glycoproteins, proteoglycans, heparan sulfate proteoglycan, glycosaminoglycans, entactin, nidogen, and peptide fragments thereof.

In various embodiments, the present cells are produced by contacting the cells with laminin α5 (e.g., without limitation, laminin 521) and one or more additional ECM agents. Such additional ECM agents include one or more laminin other than laminin α5 (e.g. one or more of laminin 111, laminin 211, laminin 121, laminin 221, laminin 332/laminin 3a32, laminin 3b32, laminin 311/laminin 3a11, laminin 321/laminin-3a21, laminin-411, laminin-421, laminin-213, and laminin-423), fibronectin (e.g. type I or II), gelatin, collagen (e.g. one or more of collagen type I, III, IV, V, and VI), and matrigel.

In various embodiments, the present methods allow for broad substrate transfer compatibility. For example, in various embodiments, the present methods allow for cells to be expanded and/or maintained on a substrate comprising the agent comprising laminin α5 (e.g. laminin 521) and transferred to another of the substrate, e.g. laminin other than laminin α5 (e.g. one or more of laminin 111, laminin 211, laminin 121, laminin 221, laminin 332/laminin 3a32, laminin 3b32, laminin 311/laminin 3a11, laminin 321/laminin-3a21, laminin-411, laminin-421, laminin-213, and laminin-423), a fibronectin (e.g. type I or II), gelatin, a collagen (e.g. one or more of collagen type I, III, IV, V, and VI), hydrogel, or matrigel, without substantial loss of differentiation capacity.

In various embodiments, the present methods allow for expansion and/or maintenance on fibronectin and differentiation when moved to a substrate comprising the one or more laminin α5 (e.g. laminin 521).

Coatings

In various embodiments, the present methods provide for coating on the surface of a cell growth substrate.

In some embodiments, the one or more laminin α5 (e.g., without limitation, laminin 521) is used to coat the surface of a substrate to promote cell adhesion to the substrate, and to stimulate cell proliferation, differentiation, and/or maintenance. The substrate used herein may be any desired substrate. For laboratory use, the substrate may be glass or plastic or other cells. For use in vivo, the substrate may be any biologically compatible material capable of supporting cell growth. Illustrative suitable substrate materials include shaped articles made of or coated with such materials as collagen, regenerated collagen, polyglycolic acid, polygalactose, polylactic acid or derivatives thereof; biocompatible metals such as titanium and stainless steel; ceramic materials including prosthetic material such as hydroxylapatite; synthetic polymers including polyesters and nylons; polystyrene; polyacrylates; polytetrafluoroethylene, and virtually any other material to which biological molecules can readily adhere.

In various embodiments, the invention provides for coating cell culture plastic or glass with human laminin 521. In some embodiments, the laminin 521 is coated at a coating concentration of about 10 ug/ml to about 20 ug/ml (e.g. about 10 ug/ml, about 11 ug/ml, about 12 ug/ml, about 13 ug/ml, about 14 ug/ml, about 15 ug/ml, about 16 ug/ml, about 17 ug/ml, about 18 ug/ml, about 19 ug/ml, about 20 ug/ml, or about 10-20 ug/ml, or about 10-17.5 ug/ml, or about 10-15 ug/ml, or about 10-12.5 ug/ml. In various embodiments, coating is conducted for about 2 hours at 37° C. or overnight at 4° C. Afterwards, laminin 521 coating solution is decanted and replaced with satellite cell growth media, e.g. DMEM/F12.

In various embodiments, the coating is in the presence of calcium and/or magnesium.

Methods of Treatment

In one aspect, the present invention relates to a method of treating a neuromuscular disease or disorder by administering muscle progenitor cells (satellite cells) of the present invention to a subject. In various embodiments, the one or more laminin α5 (e.g., without limitation, laminin 521) is used to prepare an effective amount of the progenitor cell for use as a cell-based therapy for a neuromuscular disease or disorder.

In some embodiments, a method for treating or preventing a neuromuscular disease or disorder is provided comprising preparing a progenitor cell as described above, e.g., by culturing a progenitor cell in the presence of laminin α5 and administering an effective amount of the cultured progenitor cell to a subject in need thereof.

In some embodiments, the neuromuscular disease or disorder is an injury (e.g. muscular injury) and the present cells are useful for repair.

In some embodiments, the neuromuscular disease or disorder is a myopathy.

In some embodiments, the neuromuscular disease or disorder includes muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy), motor neuron diseases (e.g. amyotrophic lateral sclerosis (ALS), Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphoglycerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenita paramyotonia congenita central core disease nemaline myopathy myotubular myopathy periodic paralysis).

In some embodiments, the neuromuscular disease or disorder is a muscular dystrophy or related myopathy, e.g. Becker muscular dystrophy (OMIM 300376, the entire contents of which are hereby incorporated by reference), congenital muscular dystrophy, Duchenne muscular dystrophy (OMIM 310200, the entire contents of which are hereby incorporated by reference and including Steinert's Disease and DM2), distal muscular dystrophy (OMIM 254130, the entire contents of which are hereby incorporated by reference), Emery-Dreifuss muscular dystrophy OMIM 310300 and 181350, the entire contents of which are hereby incorporated by reference), facioscapulohumeral muscular dystrophy (OMIM 158900, the entire contents of which are hereby incorporated by reference), limb-girdle muscular dystrophy, myotonic muscular dystrophy (OMIM160900 and 602668, the entire contents of which are hereby incorporated by reference), and oculopharyngeal muscular dystrophy (OMIM 164300, the entire contents of which are hereby incorporated by reference)).

Muscular dystrophy refers to a group of diseases that cause weakness and progressive degeneration of skeletal muscles. There are different forms of muscular dystrophy which differ in their mode of inheritance, age of onset, severity and pattern of muscles affected. The most well-known muscular dystrophies are Duchenne, Becker, limb girdle, congenital, facioscapulohumeral, myotonic, oculopharyngeal, distal, Miyoshi myopathy and Emery-Dreifuss but there are more than 100 myopathies with similarities to muscular dystrophy, all of which are included within the scope of the present invention. The term “myopathy” refers to a muscular disease in which the skeletal muscle fibers do not function for any one of many reasons, resulting in muscular weakness.

Duchenne muscular dystrophy has been often considered as a model muscular dystrophy. Duchenne muscular dystrophy results from mutations in the gene coding for the protein dystrophin, which localizes at the inner face of the sarcolemma. Dystrophin associates with a large complex of membrane proteins, called the dystrophin glycoprotein complex, important for cell membrane integrity. Without the dystrophin complex to tether the actin cytoskeleton inside the muscle cell to the extracellular matrix, forces generated by the muscle fiber result in tears of sarcolemma leading to muscle damage. The mdx mice strain is the most widely used animal model for Duchenne muscular dystrophy, having a nonsense mutation in exon 23 which eliminates dystrophin expression. Human patients with Duchenne muscular dystrophy and mdx mice suffer progressive skeletal muscle degeneration.

Muscle degeneration is a common feature of muscular dystrophy patients. Skeletal fiber loss is initially compensated by proliferation and fusion with preexisting fibers of satellite cells, resulting in an increase in muscle size. After repetitive cycles of muscle degeneration and regeneration, the dystrophic muscle damage can, however, ultimately not be repaired anymore and the dystrophic fibers become gradually replaced, initially by fibrotic infiltrates and subsequently by fat tissue. In fact, muscles of Duchenne muscular dystrophy patients or mdx mice, as well as other muscular dystrophy patients, present high fibrosis. The whole degenerative process leads to loss of normal muscle function.

Thus, in some embodiments, the present improved cells and cell productions allow for improved treatments of muscular dystrophies. The muscular dystrophy to be treated is selected from Duchenne muscular dystrophy, Becker's muscular dystrophy, limb girdlemuscular dystrophy, congenital muscular dystrophy, facioscapulohumeral muscular dystrophy, myotonic muscular dystrophy, oculopharyngeal muscular dystrophy, distal muscular dystrophy, and Emery-Dreifuss muscular dystrophy. In a particular embodiment, the treatment provided is for Duchenne muscular dystrophy.

In various embodiments, the present methods provide a reduction or alleviation of one or more of muscle pain, muscle weakness, muscle stiffness, difficulty in walking, myotonia, fatigue, scoliosis, axonal peripheral neuropathy, cardiomyopathy, cardiac arrhythmia, mental retardation, hypersomnia, sleep apnea, iridescent posterior subcapsular cataracts, insulin insensitivity, type II diabetes mellitus, premature balding, testicular failure, infantile hypotonia, and respiratory deficits.

In some embodiments, the muscle progenitor cells that are cultured are autologous (derived from and provided to the same subject) or allogenic (derived from and provided to a different subject).

In various embodiments, the present treatment methods further comprise an additional therapeutic agent which is selected based on the disease state for which the cell-based therapy is being used.

In various embodiments, the present cells are used to treat a neuromuscular disease or disorder (e.g. a muscular dystrophy, e.g. Duchenne muscular dystrophy) in combination with an additional therapeutic agent. Illustrative additional therapeutic agents include corticosteroids such as prednisone, deflazacort and VBP15. Further illustrative additional therapeutic agents include ataluren (TRANSLARNA, PTC Therapeutics), PTC 124, eteplirsen (Sarepta), SRP-4045 (Sarepta), SRP-4052 (Sarepta), SRP-4053 (Sarepta), tamoxifen, idebenone, PB1046, vamorolone, TAS-205, NS-065/NCNP-01, Rimeporide, DS-5141b, Drisapersen, FG-3019, Deflazacort, Sustanon (testosterone), BMS-986089, HT-100, CAP-1002, and CAT-1004. Further still illustrative additional therapeutic agents include supplements, such as coenzyme Q10, carnitine, amino acids (e.g. glutamine, arginine), anti-inflammatories/anti-oxidants (e.g. fish or krill oil, vitamin E, green-tea extract), and others.

In various embodiments, routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection. In various embodiments, the cells of the present invention can be administered by intravenous infusion or bolus injection. In various embodiments, the cells of the present invention can be administered by infusion or engraftment. The mode of administration can be left to the discretion of the practitioner, and depends in-part upon the site of the medical condition. In most instances, administration results in the release of any agent described herein into the bloodstream.

Dosage forms suitable for parenteral administration (e.g. intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g. lyophilized composition), which can be dissolved or suspended in sterile injectable medium immediately before use. They may contain, for example, suspending or dispersing agents known in the art.

Further, the cells described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for therapeutic use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

In some embodiments, the subject and/or animal is a human. In some embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

Drug Discovery

In various embodiments, the present invention provides for methods of drug discovery with the muscle progenitor cells (satellite cells) of the present invention. In various embodiments, the present methods provide sufficient scale-up of skeletal muscle precursors into hundreds of millions or billions of cells for high throughput drug screening. In order to perform drug discovery in relevant models derived from stem cells isolated from patients, it is critical that the cells maintain the expansion and differentiation potential of the primary cell type. Given that target cells for assays addressing the majority of neuromuscular disease are multinucleated and differentiated myotubes and not transit amplifying myogenic cells, it is critical that culture conditions support the ability of the expanded primary cells to differentiate effectively into myotubes. Accordingly, the present invention allows for such drug discovery methods.

Cells made according to the methods and compositions described here may be used to screen for factors (such as solvents, small molecule drugs, peptides, polynucleotides, and the like) or environmental conditions (such as culture conditions or manipulations) that affect the characteristics of differentiated cells.

In some applications, the present cells are used to screen factors that promote maturation, or promote proliferation and maintenance of such cells in long-term culture. For example, candidate maturation factors or growth factors are tested by adding them to progenitor cells or differentiated cells in different wells, and then determining any phenotypic change that results, according to desirable criteria for further culture and use of the cells.

Furthermore, gene expression profiling of the present cell may be used to identify receptors, transcription factors, and signaling molecules that are unique or highly expressed in these cells. Specific ligands, small molecule inhibitors or activators for the receptors, transcription factors and signaling molecules may be used to modulate differentiation and properties of progenitor cell lines and differentiated cells.

Particular screening applications relate to the testing of pharmaceutical compounds in drug research. For instance, the methodologies in In vitro Methods in Pharmaceutical Research, Academic Press, 1997 (Eds. Castell and Gómez-Lechó) and U.S. Pat. No. 5,030,015, the entire contents of which are incorporated by reference in their entireties, provide various drug discovery methods which are applicable to the present invention.

In various embodiments, assessment of the activity of candidate pharmaceutical compounds generally involves combining the cells with the candidate compound, determining any change in the morphology, marker phenotype, or metabolic activity of the cells that is attributable to the compound (compared with untreated cells or cells treated with an inert compound), and then correlating the effect of the compound with the observed change.

The screening may be done, for example, either because the compound is designed to have a pharmacological effect on certain cell types, or because a compound designed to have effects elsewhere may have unintended side effects. Two or more drugs can be tested in combination (by combining with the cells either simultaneously or sequentially), to detect possible drug-drug interaction effects. In some applications, compounds are screened initially for potential toxicity (see, e.g., In vitro Methods in Pharmaceutical Research, Academic Press, 1997 (Eds. Castell and Gómez-Lechó), pp. 375-410, the entire contents of which incorporated by reference in its entirety). Cytotoxicity can be determined in the first instance by the effect on cell viability, survival, morphology, and expression or release of certain, markers, receptors or enzymes. Effects of a drug on chromosomal DNA can be determined by measuring DNA synthesis or repair. [3H]thymidine or BrdU incorporation, especially at unscheduled times in the cell cycle, or above the level required for cell replication, is consistent with a drug effect. Unwanted effects can also include unusual rates of sister chromatid exchange, as determined by metaphase spread.

In various embodiments, the present cells are useful in drug discovery efforts for agents that are beneficial for muscle health, e.g. those which may be used to treat or prevent one or more neuromuscular disease or disorder.

Kits

The invention provides kits that can simplify the cell production or treatment methods described herein. An illustrative kit of the invention comprises any composition, including produced cells, described herein in unit dosage form. The kit may comprise progenitor cells in a medium suitable for culturing and laminin α5. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

In one embodiment, the kit can comprise articles for cell culture, such as a cell growth substrate that is optionally pre-coated with agents described herein and culture media.

This invention is further illustrated by the following non-limiting examples.

### EXAMPLES

Materials and Methods

Isolation and Culture of Murine Satellite Cells

Primary murine satellite cells were isolated from tibialis anterior and quadriceps muscles from 12 week old DBA/2J male mice. Dissected muscles were minced with scalpel blades and digested in DMEM/F12 (Life Technologies, 1:1 mixture) containing 2% collagenase II (Worthington Biochemicals) and 1.2 U/ml dispase (Worthington Biochemicals) with 2.5 mM CaCl₂. Digestions were incubated at 37° C. for 1 hour with trituration and mixing every 15 minutes. Cells were filtered through 100 μM and 40 μM cell strainers (BD). Cells were pelleted by centrifugation for 5 min at 300 g. Cells were resuspended in FACS staining buffer (DMEM/F12/0.5% BSA/25 mM HEPES) and distributed in 200 μl aliquots into staining tubes. Cells were blocked using anti-CD16/CD32 antibody (Ebioscience) at 1:100 dilution for 10 min on ice. Cells were stained with the following antibodies on ice for 30 min: CD31-FITC (1:50, Ebioscience, 390) CD45-FITC (1:50, Ebioscience, 30-F11), PDGFRα-BV421 (1:40, BD, APAS), Scal-BV605 (1:100, BD, D7), and Integrin α7 (1:400, Ablab, R2F2). Cells were washed twice with sort buffer (HBSS/0.5% BSA/25mM HEPES) including centrifugation for 5 min at 300 g. Compensation controls were prepared using Ultracomp beads (Ebioscience). Single only bead controls were stained in 100 μl with 2 μl of each antibody for 15 min at room temperature. Beads were washed once with sort buffer and resuspended in sort buffer. Compensation was calculated using single stained and unstained bead controls with FACS DIVA compensation wizard. Gating was determined by using fluorescence minus one plus isotype controls. Dead cells were gated out using propidium iodide (Life Technologies).

ECM Coating and Culture

Laminins including laminin 111, laminin 211, laminin 332, laminin 411, laminin 421, laminin 511, and laminin 521 are human recombinant isoforms obtained from Biolamina. Laminins were diluted at a concentration of 10 ug/ml in HBSS with calcium and magnesium, and coated overnight at 4° C. Fibronectin was from human placenta (Corning #354008), and coated at 10 μg/ml in distilled water for 1 hour at room temperature. Growth factor reduced MATRIGEL (Corning) was diluted 1:5 with DMEM/F12 media and thinly coated by covering plastic, removing excess, and drying matrigel for 20 minutes at 37° C.

For initial characterization, cells were plated at a density of 2,000 cells per well in 96 well format in DMEM/F12/20% FBS/Primocin (Live Technologies/ Invivogen) with 10 ng/ml mouse FGF-2 (R&D). Media was replaced after 5 days and refreshed every 3 days afterwards. To induce differentiation at day 8, media was switched to Differentiation Media (DM), DMEM/F12/5% HI-HS/Primocin, and maintained until day 11.

For long term growth, cells were plated at a density of 10,000 cells per well in 6 well format. Cells were grown in growth media as previously described and refreshed every 3-4 days with growth media and 10 ng/ml FGF-2. Cells were split using Accutase and maintained on the same substrate for 6-8 passages. To assay differentiation, cells were split using Accutase and seeded in 96 well format at a density of 4,000 cells per well. Cells were grown in GM for 5 days, and then switched to DM for an additional 5 days.

For ECM substitution experiments, cells were thawed, expanded and passaged twice before analysis. At second passage, cells were transferred to a 96 well plate containing five of the ECM substrates (laminin 111, laminin 211, laminin 521, FN, and MG). Cells were grown and differentiated similarly to the previously mentioned long term growth procedure.

Immunocytochemistry and Imaging

Immunostaining was performed in black Corning 96 well plates. For myosin heavy chain (MHC) staining, cells were fixed using Cytoperm/Cytofix for 15 min at room temperature. Cells were rinsed twice and then subsequently blocked using 10% HI-HS/0.1% Triton for 1 hour at room temperature. Cells were stained with MHC-Alexa488 antibody at 1:100 overnight at 4° C. Cells were rinsed 4 times with PBS and stained with Hoechst to identify nuclei. Images were acquired using a 10× objective on a Cellomics ArrayScan. Analysis was performed using the Cellomics HCS Studio Version 6.5 software analyzing MHC positive cells containing 2 or more nuclei. Software algorithm used was the “myotube formation” package using dynamic thresholding, 3 sigma or isodata, for myotube identification.

For Pax7/MyoD staining cells were fixed using foxp3/ki67 nuclear fixation buffer (Ebioscience) for 15 min at room temperature. Cells were rinsed twice and blocked with Block Aid (Life Technologies) for 1 hour at room temperature. Pax7 (1:50, R&D) and MyoD (1:50, 5F11, Millipore) were coincubated overnight at 4° C. in Block Aid. Cells were rinsed 3× and secondary antibodies (donkey anti-mouse Alexa488, donkey anti-rat Alexa647; 1:200) were incubated for 1 hour at room temperature. Cells were rinsed 4× and stained with Hoechst for nuclei identification. Images were acquired using a 20× objective on a Cellomics ArrayScan. Analysis was performed using the nuclear colocalization algorithm (Cellomics HCS Studio 6.5) analyzing proportion of Pax7 or MyoD positive nuclei.

Integrin FACS Analysis

Passage 8 mouse satellite cells were split using Accutase, collected and centrifuged at 300 g for 5 min, and resuspended in FACS staining buffer. Cells were blocked with FC block (BD biosciences) at 1:50 for 10 min on ice. Afterwards cells were stained with the following PE conjugated antibodies: integrin alphal (BD 562115) at 1:40, integrin alpha2 (Ebioscience 12-5971-81) at 1:40, integrin alpha3 (R&D FAP2787P) at 1:10, integrin alpha4 (Ebioscience 12-0492-81) at 1:20, integrin alpha5 (BD 553930) at 1:40, integrin alpha6 (Ebioscience 12-0495-81) at 1:200, integrin alpha7 (Ablab) at 1:200, integrin alphaV (Ebioscience 12-0512-82) at 1:50, integrin beta1 (Ebioscience 12-0291-81) at 1:20, integrin beta2 (Ebioscience 12-0181-81) at 1:20, integrin beta3 (Ebioscience 12-0611-81) at 1:40, integrin beta4 (R&D FAB4054P) at 1:20, and integrin beta5 (Ebioscience 12-0497-41) at 1:20. Cells were stained for 30 min on ice followed by two washes in FACS stain buffer. Cells were resuspended in 300 μl of FACS buffer and analyzed on the FACS Aria II. Gating was set according to negative unstained and isotype control Rat IgG2a K-PE (Ebioscience 12-4321-81).

Human Myogenic Cell Isolation

Post-mortem non-diseased skeletal muscle gracillus tissue was obtained through Asterand. Muscle was trimmed of fat and connective tissue. Tissue was minced for approximately 10 minutes. Tissue was digested using Collagenase II (Worthington Biochemicals) and Dispase (Worthington Biochemicals), for approximately 75 minutes at 3° C. Digestions were performed in gentleMACS™ Dissociators. Tissue was pulsed every 15 minutes. Following digestion, cells were strained through 100 μM, 70 μM, and 30 μM cell strainers (Miltenyi), respectively. Cells were resuspended in approximately 200 μl of MACS stain buffer (Miltenyi). Cells are stained for 1 hour on ice with the following antibodies: CD11b-FITC, Miltenyi Biotec, Catalog Number:130-081-201, CD31-FITC, Miltenyi Biotec, Catalog Number; 130-092-654, CD45-FITC, Miltenyi Biotec, Catalog Number: 130-080-202, CD34-APC, BD Biosciences, Catalog Number: 560940, CD56-PE, Miltenyi Biotec, Catalog Number; 130-090-755. Afterwards cells were rinsed twice and subsequently incubated with anti-FITC microbeads (Miltenyi Biotec, 130-048-701) for 30 min on ice followed by two washes. Afterwards, cells were passed through a Miltenyi magnetic depletion column. The column binds magnetically labelled FITC+ cells (CD31, CD45, CD11b) while allowing FITC− cells to flow through. Cells moved passively through the column into a collection tube. Afterwards cells were centrifuged, resuspended in FACS buffer, and FACS sorted (FACS ARIA II) for CD56+, CD34−, CD45−, CD31−, CD11b− cells. Myogenic cells were grown in growth media DMEM/F12 (Gibco) supplemented with 20% FBS (Gibco)/Primocin and 10 ng/ml human FGF2 (R&D). For differentiation of human cells, cells were seeded at a density of 16,000 cells per well in 96 well format. After 3 days, half of the media was replaced with differentiation media consisting of DMEM/F12 supplemented with 5% HS-HI (Gibco) and Primocin. Afterwards, half of the media was replaced every other day until day 11 when cells were fixed with Ctyoperm/Cytofix (BD).

Statistics

Statistics for multiple comparisons were conducted using one-way ANOVA with Bonferroni correction. Significance is annotated as less than 0.05(*), less than 0.01(**), less than 0.001 (. ***), and less than 0.0001 (****). All comparisons were conducted using laminin 521 as control. Significance for myotube nuclei distribution was determined using linear regression. Statistical calculations were conducted using Graphpad Prism 6.

Short-Term and Long-Term Myogenic Cell Analysis

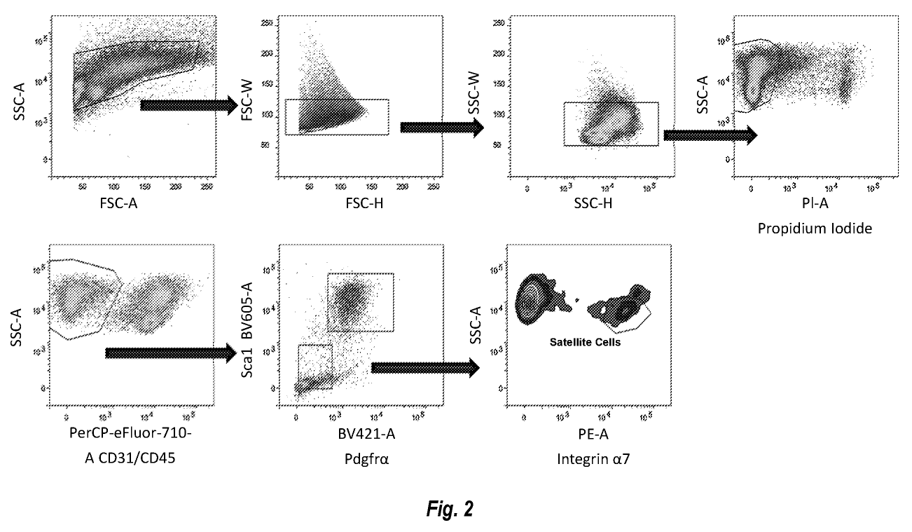

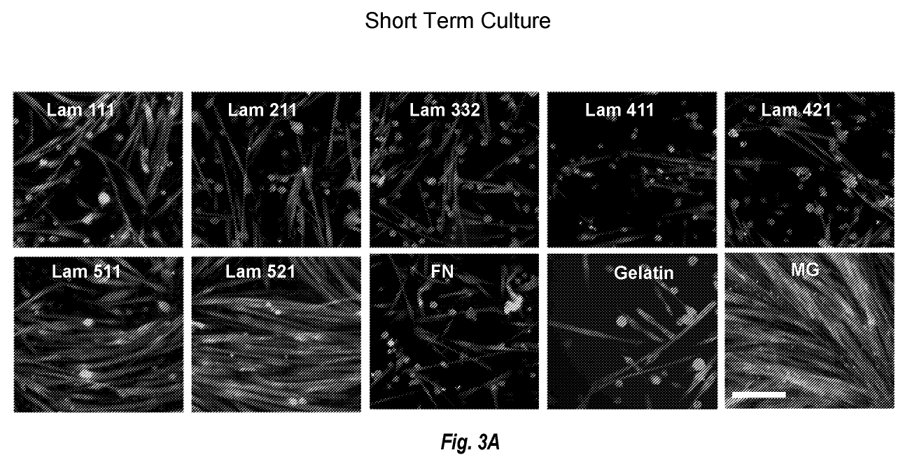

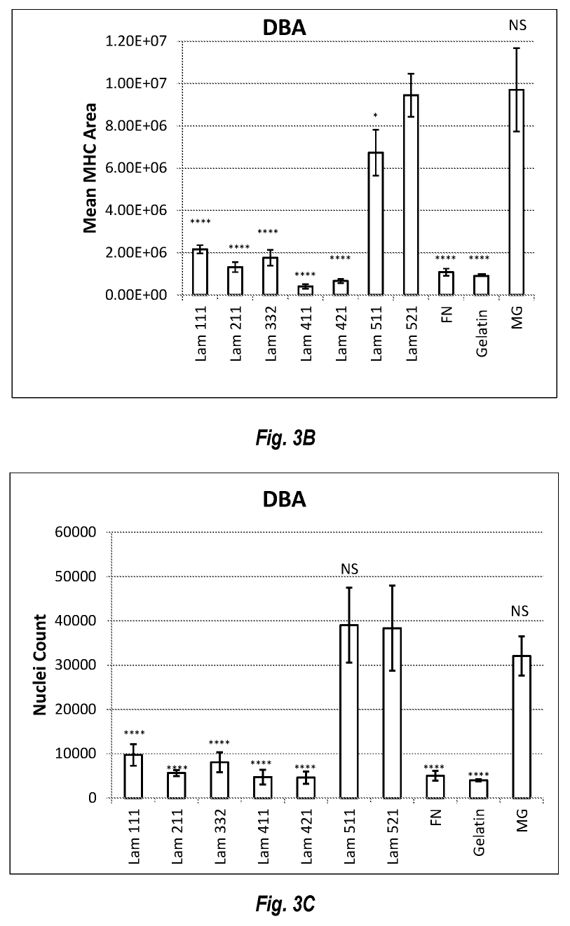

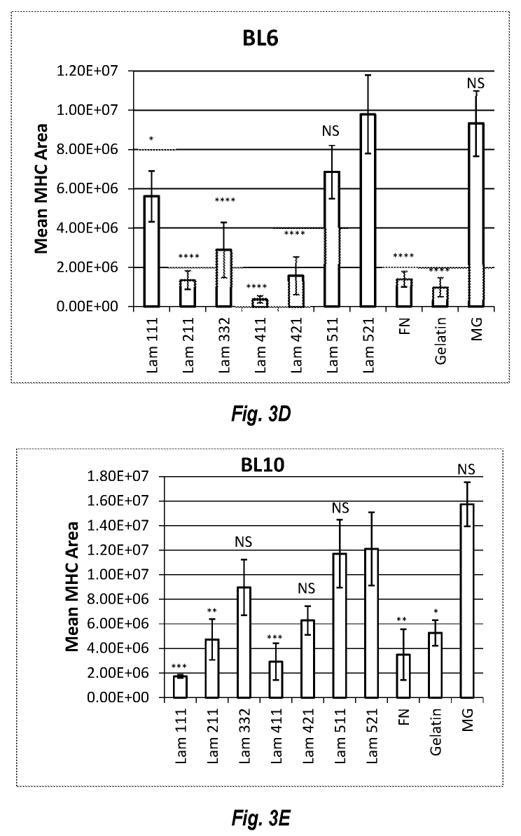

To compare the activity of freshly isolated mouse satellite cells, Integrinα7+/PDGFRα−/Sca1−/CD31−/CD45− cells were FACS sorted (FIG. 2) and plated on ECM substrates including laminin 111, laminin 211, laminin 332, laminin 411, laminin 421, laminin 511, laminin 521, fibronectin (FN), gelatin, and growth factor reduced MATRIGEL (MG) (FIG. 3A). A striking increase in proliferation resulting in a three- to four-fold increase in cell number on laminin 511, laminin 521, and MG compared to all other substrates (FIGS. 3A and 3C) was observed. In addition, cells expanded on laminin 511, laminin 521, and MG, showed dramatically enhanced differentiation as quantified by Myosin Heavy Chain (MHC) positive area (FIGS. 3B). On the other hand, cells on laminin 111 differentiated moderately while cells on laminin 211, laminin 332, laminin 411, laminin 421, and FN differentiated poorly (FIGS. 3A and 3BB). Additionally, myotubes formed on laminin 521 and MG visually appeared to be wider in appearance and overall more robust. Cells differentiated on laminin 521 appeared to be more organized in comparison and more mature as we noted the nuclei in laminin 521 cultures evenly spaced and distributed while MG myotubes nuclei had a clustered appearance and myotubes appeared less organized. To extend these results beyond the Dba mouse model, the differentiation experiments were repeated on C57/BL6 and C57/BL10 satellite cells. Consistent with the Dba satellite cell results, cellular differentiation is consistently increased on laminin 511, laminin 521, and MG compared to all other substrates (FIG. 3D for C57/BL6 satellite cells, and FIG. 3E for C57/BL10 satellite cells).

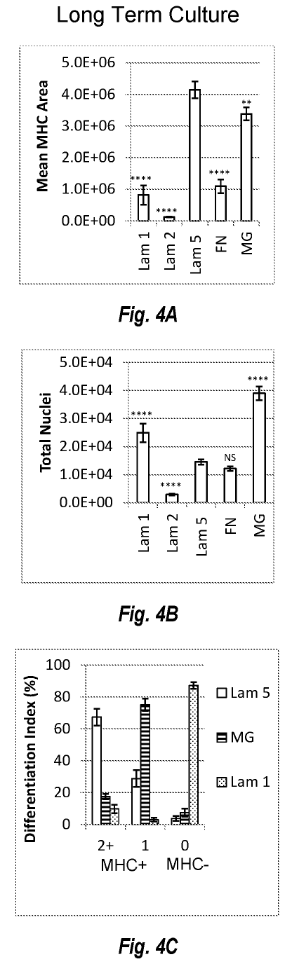

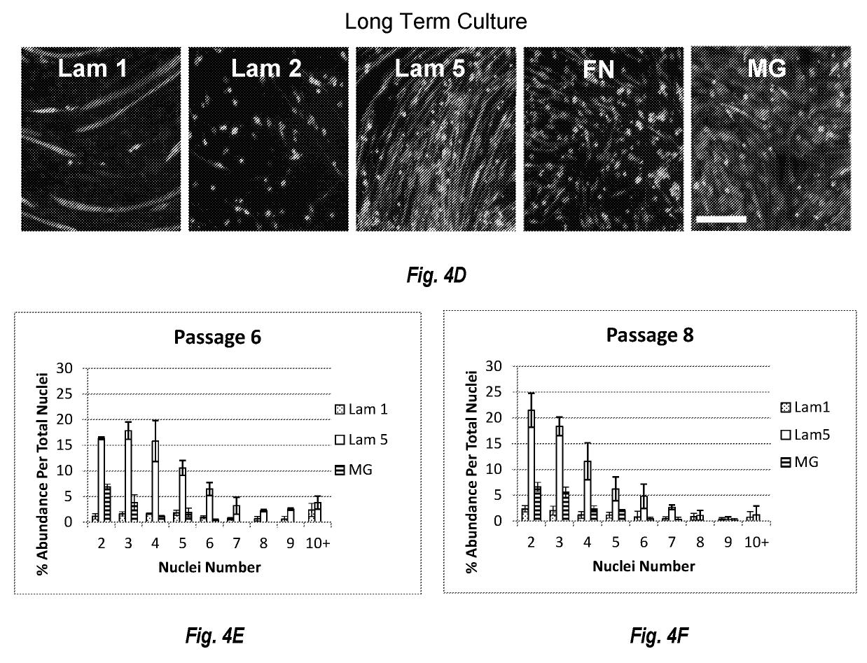

While satellite cells typically have strong differentiation potential when freshly isolated, no study has evaluated a variety of ECMs to expand satellite cells to maintain their stem cell and differentiation potential after long term culture. Therefore laminin 111, FN, and MG due to their common usage in the literature were selected, as well as laminin 211 and laminin 521 due to their expression in vivo. Additionally, laminin 521 was selected over laminin 511 due to the observed performance benefit in the short term study in FIGS. 3A-3D. Cells were grown for 6-8 passages and then assayed for proliferation and differentiation. Similar to the short-term results significant differences in differentiation among different ECMs were found, and these differences appear to be amplified over the long term. Laminin 111 displayed significant proliferation but differentiated minimally (FIGS. A and 4D). While the myotubes formed on laminin 111 were fairly large in size, the majority of the cells in culture were negative for MHC (FIG. 4C). Laminin 211 on the other hand performed similarly to the fresh analysis from FIGS. 3A-3D where cells expanded at a very slow rate and failed to differentiate (FIGS. 4A and 4D). FN expanded cells differentiated minimally resulting in very thin and small myotubes (FIGS. 4A and 4D). Laminin 521 and MG both were the only substrates that supported extensive myogenic differentiation, as assayed by MHC positive area after culture in differentiation media (DM) (FIGS. 4A and 4D). However, while Laminin 521 and MG have similar MHC area percentages, cells on Laminin 521 form more multinucleated myotubes, defined as myotubes containing 2 or more nuclei, compared to cells on MG (FIG. 4C). Moreover, MG cells up-regulate MHC but fail to fuse significantly remaining in a myocyte stage resulting in approximately 70% of the cells expressing MHC but only containing one nucleus (FIG. 4C). On the other hand, 70% of laminin 521 assayed cells contain 2 or more nuclei (FIG. 4C). To further quantify myogenic differentiation an in depth multi-nucleation index was performed to quantify the number of nuclei per myotube proportional to total nuclei, of myotubes on laminin 111, laminin 521, and MG. At both passage 6 and passage 8, laminin 521 myotubes contained increased proportions of nuclei per myotube compared to MG and laminin 111 (FIGS. 4E and 4F). Strikingly, laminin 521 myotubes contained a broad increase in the proportion of nuclei per myotube over the entire distribution of myotubes ranging from 2-10 nuclei per myotube (FIGS. 4E and 4F). Overall these results reveal that Laminin 521 is a superior substrate for expanding myogenic cell cultures over long-term passage while maintaining excellent differentiation.

ECM Exchange

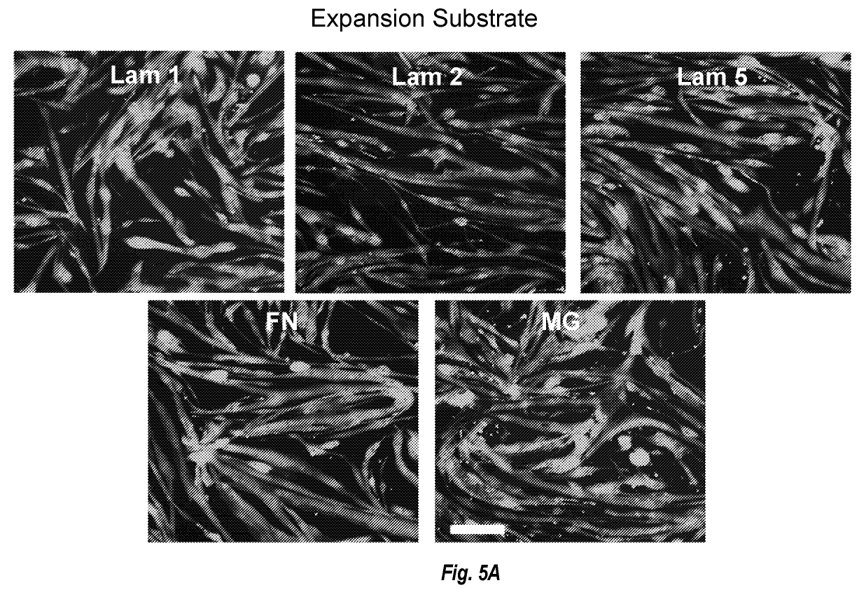

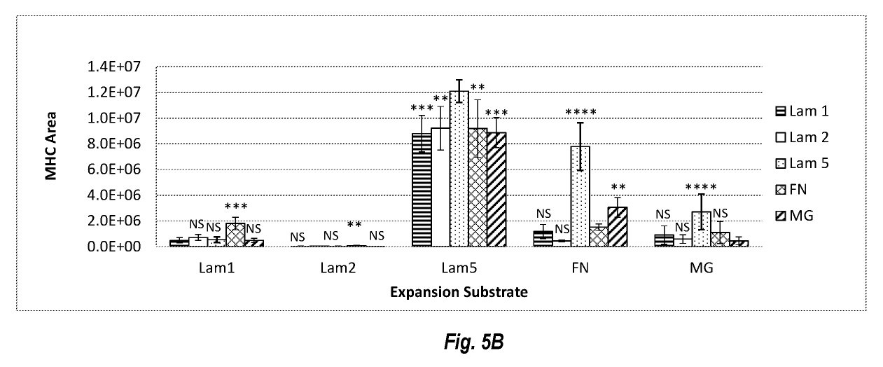

To determine the utility of using laminin 521 for cell types already expanded on substrates other than laminin 521, a substrate substitution experiment was performed evaluating previously isolated primary mouse satellite cells on each of the other substrates in the study. Cells expanded on laminin 521 show robust differentiation when transferred to any of the substrates tested here including laminin 111, laminin 211, FN, and MG (FIGS. 5A and 5B). In comparison, while cells maintained on laminin 521 demonstrated the highest differentiation performance, cells moved from laminin 521 to other substrates (laminin 111, laminin 211, FN, and MG) showed a small reduction in differentiation (FIGS. 4A and 4B). Additionally, we observed a lag in initial proliferation when laminin 521 cells were transferred to other substrates, although cultures did gradually increase proliferation over time (data not shown). The only additional expansion substrate showing substrate-substrate compatibility were cells expanded on FN; cells showed significant differentiation when moved to laminin 521 (FIG. 5B). Cells expanded on all other substrates, including laminin 111, laminin 211 and MG, failed to differentiate significantly, both on their original substrates and when moved to other substrates (FIG. 5B). These results reveal that laminin 521 expanded satellite cells demonstrate superior propensity to maintain differentiation and reveal unique, broad, substrate transfer compatibility.

Marker Analysis

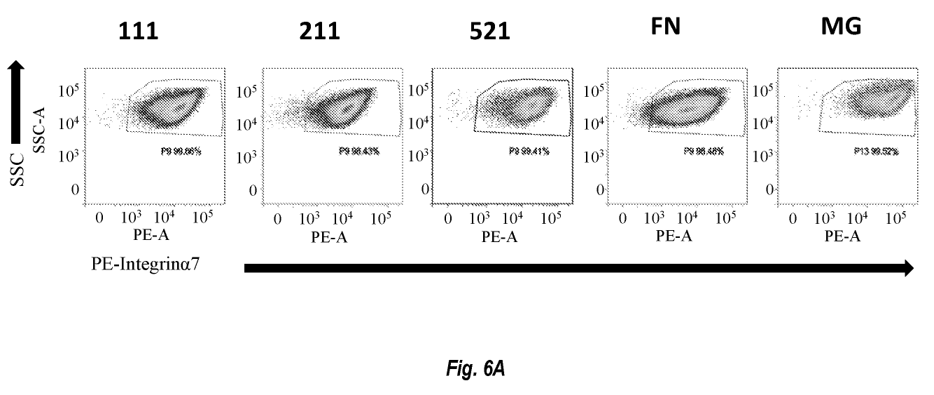

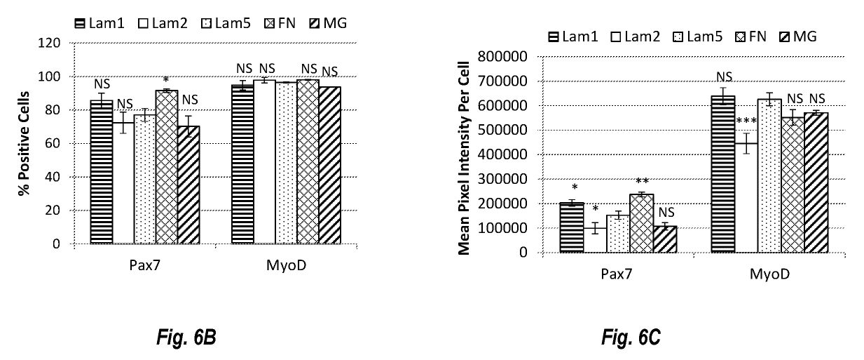

Due to the large differences observed in myogenic cell performance a set of control experiments to rule out the presence of contaminating non-myogenic cells in the primary mouse satellite cell cultures was performed. FACS staining revealed 99% of cells stained positive for integrin α7 (FIG. 6A) while there were no detectable PDGFRα or CD31 positive cells present (data not shown) suggesting that the cultures were homogenous for myogenic cells. Subsequently, cells from passage 6 were immunostained, during the expansion phase in growth medium, to determine the expression of Pax7 and MyoD to assess if changes in their expression or intensity may explain the dramatic difference in myogenic activity on different ECMs (FIGS. 6B and 6C). Similar proportions of Pax7 and MyoD positively stained cells on each substrate were observed with the exception of an increased proportion of Pax7 positive cells expanded on FN (FIG. 6B). Protein staining intensity level varied minimally for Pax7 and MyoD expression (FIG. 6C). Statistically significant differences for pax7 expression level changes were observed, however changes were small ranging around +/−50% compared to laminin 521 (FIG. 6C). MyoD expression levels were consistent except for a reduction in expression level in laminin 211 cultures. Interestingly, Scal expression was found to be present in a significant proportion of cells cultured on laminin 111, laminin 211, and FN but was absent in cultures maintained on laminin 521 and MG (data not shown). These results agree with previous studies in which case Scal expression on satellite cells was associated with cells exhibiting poor differentiation. These results suggest that Scal may be a marker associated with differentiation deficient myogenic cells; however, this will require further study.

Integrin Profiling

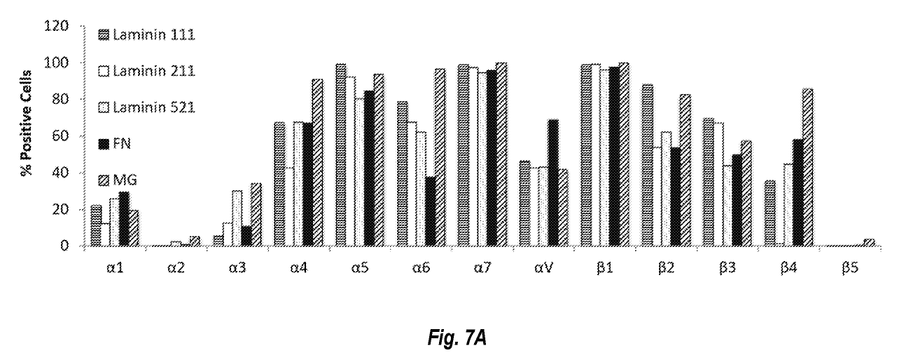

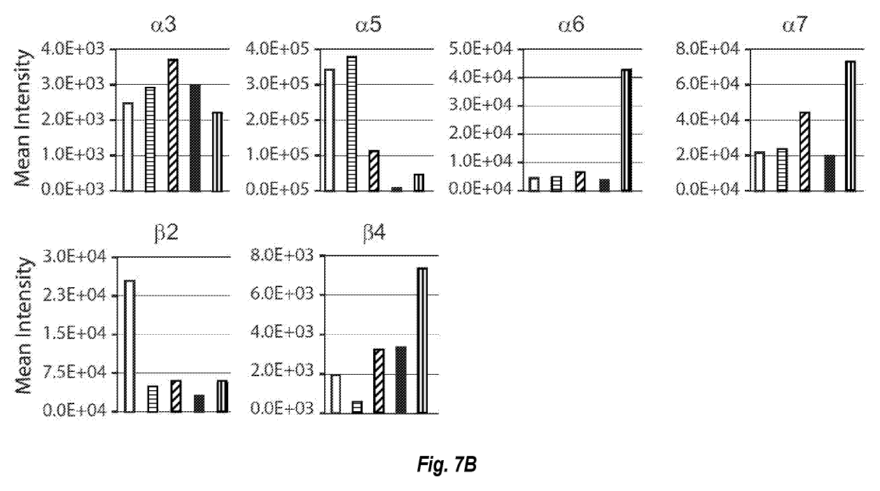

Integrin receptor signaling plays many critical roles during myogenesis. Since laminins, FN, and components of MG, activate many of their functions via integrin receptors, it was hypothesized, without wishing to be bound by theory, that the observed differences in long term culture may be caused by shifts in integrin expression on different ECMs. Previously expanded mouse cells were assayed at passage 8 in growth conditions on each ECM (laminin 111, laminin 211, laminin 521, FN, and MG) by FACS staining using integrin α1-7, integrin αV, and integrin β1-5 antibodies (FIG. 7A). Close to 100% of cells grown on all substrates expressed integrin α7 and β1 (Figure A). On the other hand, only a small proportion of cells expressed al (10-20%) while less than 5% of cells on any substrate expressed α2 or β5 (FIG. 7A). Meanwhile, the remaining integrins showed some degree of heterogeneous expression across substrates. Expression of α4, α5, β2, and β4 was similar and heterogeneous on most substrates, with the exception of the high expression of β2 on laminin 111 and MG, and an absence of expression of β4 on laminin 211 and elevated expression on MG (FIG. 7A). Interestingly, integrin α3 was expressed by a larger proportion of cells, approximately 30%, on cells expanded on laminin 521 and MG while it was expressed by less than 10 percent on cells expanded on all other ECMs (FIG. 7A). In addition to population changes variations in mean fluorescent intensities for a subset of integrins including integrin α3, integrin α5, integrin α6, integrin α7, integrin β2, and integrin β4 were also observed (FIG. 7B). For example, integrin α3 showed elevated expression on laminin 521 expanded cells. Integrin α5 showed elevated expression in cells grown on laminin 111 and laminin 211, while integrin β2 had highest expression on laminin 111. Integrin α6 expression was increased dramatically on MG cultured cells while integrin α7 was expressed higher on MG cultured cells and, to a lesser extent, on laminin 521 cells. Lastly, integrin β4 showed very little expression on laminin 211 cells while it was up-regulated in MG cultured cells. Taken together these results suggest that both the proportion of cells expressing each integrin and the expression level of integrins varies with different ECM matrices. Moreover, due to the complexity observed here, without wishing to be bound by theory, there are likely multiple mechanisms contributing to the different characteristics of cells expanded on different ECMs.

Human Satellite Cell Culture and Expansion

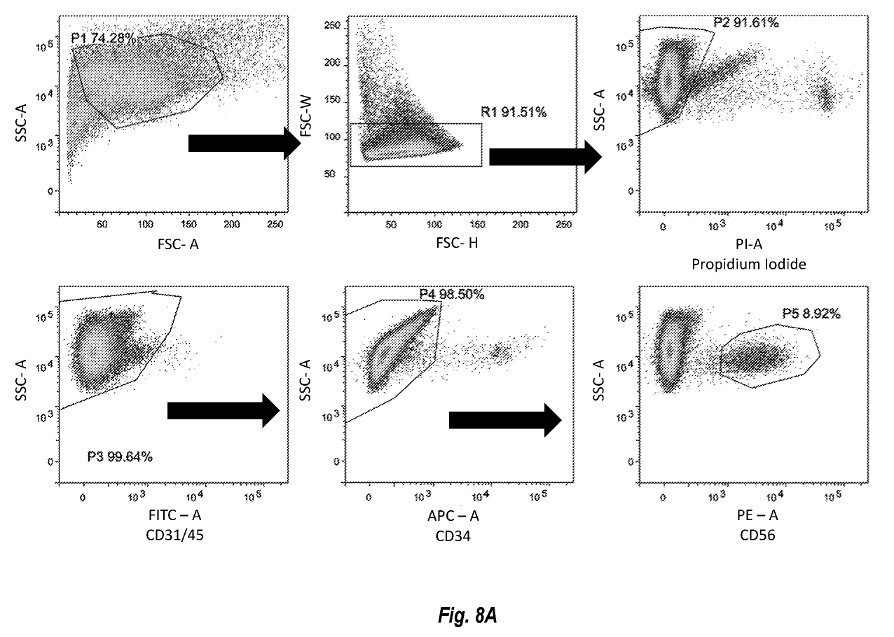

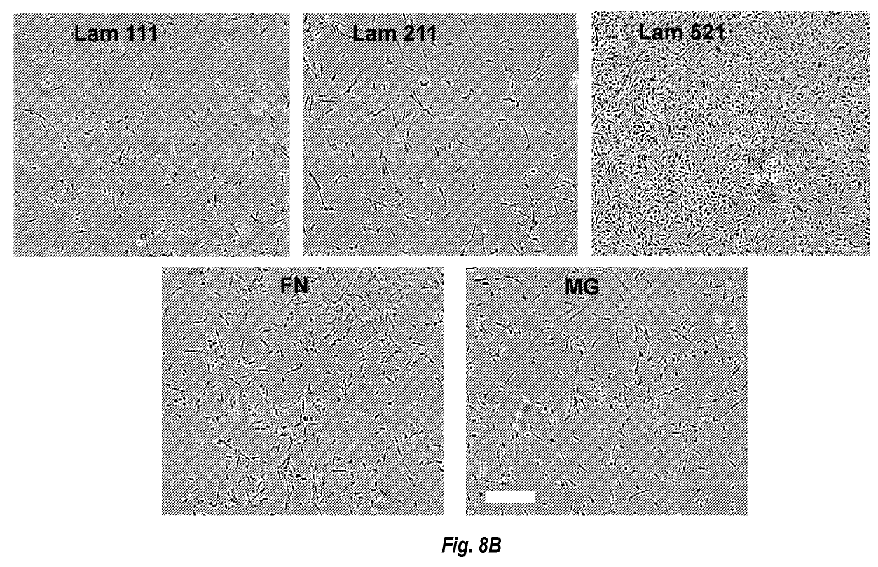

In an effort to determine if the result obtained with the mouse model translates to humans, a similar evaluation was undertaken with freshly isolated human muscle cells. Human satellite cells were isolated from the gracilis muscle obtained from a post-mortem patient lacking diagnosed skeletal muscle disease. CD56+/CD31−/CD45−/CD11b− satellite cells were isolated using a dual MACS/FACS approach (FIG. 8A). Cells were initially expanded on each of the following substrates: laminin 111, laminin 211, laminin 521, FN, and MG. Within the first week of growth a dramatically increased growth rate with cells expanded on laminin 521 was observed compared to all other substrates (FIG. 8B). Additionally, laminin 211 appeared to display a lag in cell growth compared to other substrates, similar to earlier satellite cell findings in mouse (FIG. 8B).

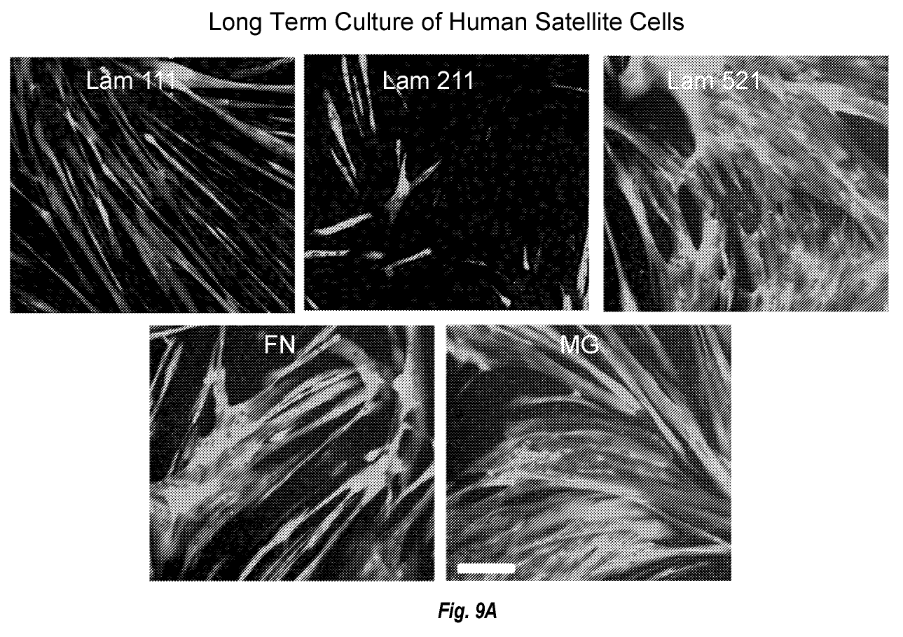

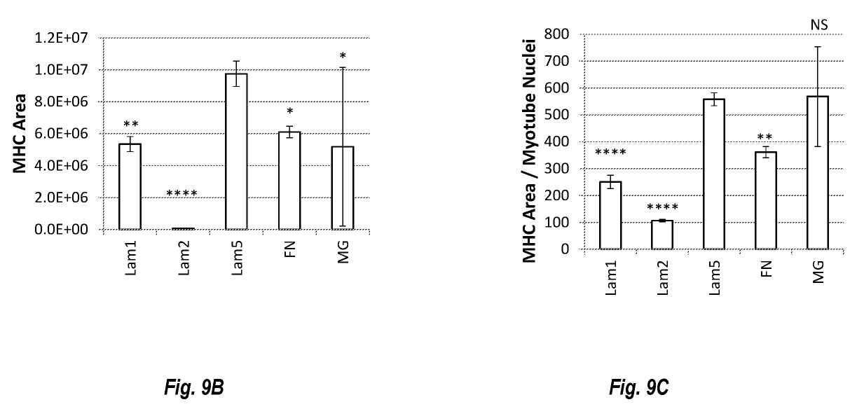

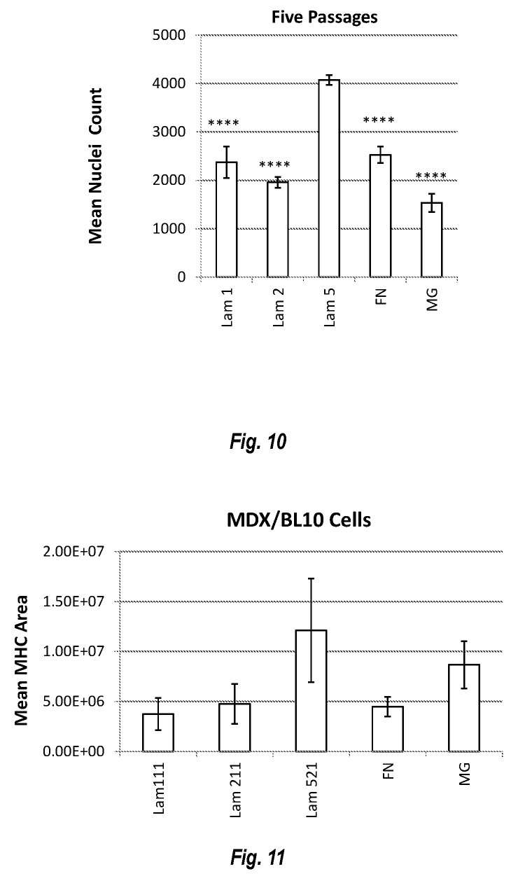

Since the mouse studies revealed unexpected expansion and differentiation effects on long term culture of satellite cells on specific substrates, we next performed a similar study on the newly generated human satellite cells. Human satellite cells expanded at a faster rate on laminin 521 compared to other substrates following 5 passages (FIG. 10). In addition, the human cells displayed the highest amount of differentiation on laminin 521 and MG, followed by moderate differentiation on laminin 111 and FN substrates, and poor differentiation on laminin 211 (FIGS. 9A and 9B). In addition, myotubes formed on laminin 521 and MG appeared to be hypertrophic due to an increased amount of MHC area staining in proportion to myotube nuclear count (FIG. 9C). Importantly, it was observed that differentiated myotubes maintained better attachment on laminin 521 compared to MG, in which case larger variability on MG due to myotube detachment was observed. Laminin 211 differentiated cultures performed poorly, similar to the observations with the mouse cultures. This resulted in a majority of cells staining negative for MHC expression and a very small MHC area value (FIGS. 9B and 9C). Laminin 111 and FN cultures differentiated well but only reached approximately half of the MHC area compared to laminin 521 or MG cultures (FIG. 9B).

In addition to the mouse study presented here, an analysis of freshly isolated mdx/BL10 cells was performed, and a very similar pattern with laminin 521 outperforming laminin 111, laminin 211, FN, and MG in differentiation was observed (FIG. 11). Furthermore, difficulty in expanding mdx/BL10 cells on laminin 111 were not encountered on laminin 521 (data not shown). Importantly the findings translate to human myogenic cell culture as the human cells perform exceptionally on laminin 521 showing superior proliferation and differentiation to all other substrates tested. Taken together the results demonstrate laminin 521 as superior substrate for satellite cell expansion while demonstrating translatability across several mouse backgrounds (Dba/2J, C57/BL10), human cells, and with disease states (mdx/BL10).

Engraftment

Muscle stem cells were isolated from pax7 reporter mice expressing firefly luciferase (Pax7 Rydl satellite cells, Yfp/luci/DTR). Isolation was performed using enzymatic digestion and fluorescent activated cell sorting using muscle stem cell specific antibody integrin α7. Cells were passaged on each substrate independently on laminin 111, laminin 521, and growth factor-reduced matrigel (MG). Laminin coatings were prepared by coating cell culture ware with 10 μg/ml laminin in HBSS containing calcium and magnesium overnight at 4° C. MG was prepared by diluting 1:5 in serum-free media and thin coating plastic ware followed by drying at 37° C. for 30 min. Initial seeding was performed in 6-well plate format. Cells were expanded and split when cells reached approximately 60-75% confluency. Cells were split using Accutase enzymatic solution. Cells were frozen at passage 6. Cells were thawed and passaged two additional times on the same corresponding substrates as their initial expansion. To prepare the skeletal muscle for engraftment experiments, limb muscles (tibialis anterior, gastrocnemius, and quadriceps) of recipient mice were injected with 75 μl of 10 μM cardiotoxin 3 days prior to cellular injection. Cardiotoxin injection induces muscle degeneration and subsequent muscle regeneration. On day 3 following cardiotoxin injection, cells were lifted with Accutase, counted, and resuspended in 0.5% BSA DMEM/F12 with 25 mM HEPES/Phenol Red-free media (Gibco). Mice were anesthetized using Isoflurane gas. 100,000 cells were injected intramuscularly into the left gastrocnemius and quadriceps, and 50,000 cells were injected into the left tibialis anterior. Volume for tibialis anterior is 50 μl, gastrocnemius and quadriceps received 100 μl each.

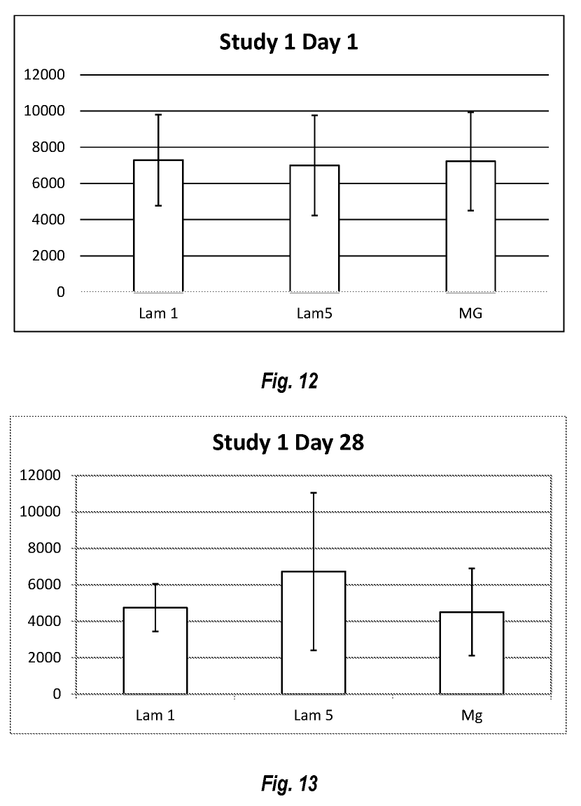

The first engraftment study followed 18 mice (6 per substrate group) for 28 days. At Day 1 post-injection, mice were imaged in a LagoX live animal imager. Mice were injected via intraperitoneal route with 200 μl of Rediject Luciferin-D to visualize luciferase (FIG. 12). The Y-axis units show relative light units. At the conclusion of the study, increased luciferase signal in laminin 521 engrafted cells was observed as compared to both laminin 111 and MG expanded cells (FIG. 13). There were issues with anesthesia, as 5 animals died during anesthesia.

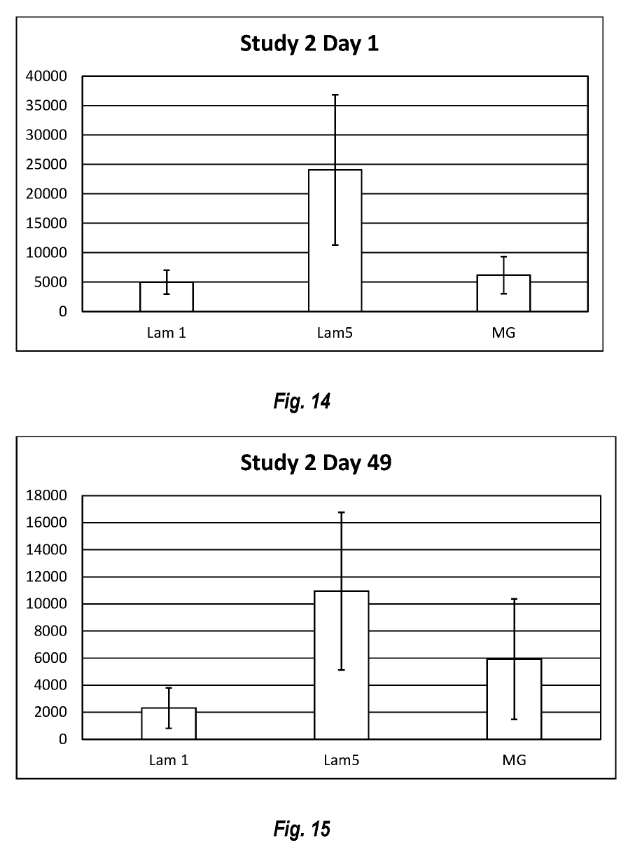

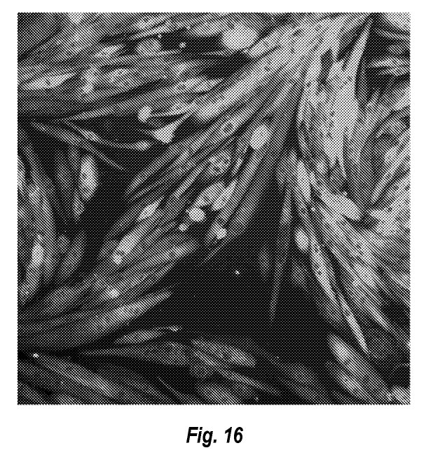

The second engraftment study followed 30 mice (10 per substrate group) for 49 days (2 mice in the MG group passed before day 49) using the same methods described above. At Day 1 post-injection, mice were imaged in a LagoX live animal imager. Mice were injected via intraperitoneal route with 200 μl of Rediject Luciferin-D to visualize luciferase (FIG. 14). The Y-axis units show relative light units. At the conclusion of the study, increased luciferase signal in laminin 521 engrafted cells was observed as compared to both laminin 111 and MG expanded cells (FIG. 16). These results demonstrate that the laminin 521 substrate may be used to increase the engraftment potential of cultured muscle cells and improve maintenance of engrafted cells for an extended period of time.

Long-Term Passage

Satellite cells previously cultured for 14 passages on laminin 521 were passed one additional time to a total of 15 passages and differentiated in differentiation media (DMEM/F12+5% HI-HS+Primocin). Cells were seeded at density of 10,000 cells per well and media was changed 50% every two days. After 10 days in differentiation conditions, cells were fixed and analyzed for MHC expression to quantify myotubes. Cells were fixed using 4% PFA for 15 minutes, two washes in PBS, staining with Myosin Heavy Chain antibody at 1:100 dilution (Ebioscience) overnight at 4° C., followed by 4 washes in PBS, staining with Donkey-anti-mouse (1:400 dilution) Rhodamine Red labeled for 1 hour at room temperature, 4 washes in PBS, stain with Hoecsht 1:10,000 dilution for 5 min, wash out with PBS once. Imaging was conducted with Arrayscan VTi (FIG. 16). After 15 passages, cells maintained their ability to differentiate and numerous MHC containing myotubes containing 2 or more nuclei were observed. Culturing with laminin 521 thus demonstrated improved differentiation results with multiple myotubes with 4 or more nuclei after 15 passages.

Other than in the examples herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (e.g., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated.

While this invention has been particularly shown and described with references to preferred embodiments thereof, it will be understood by those skilled in the art that various changes in form and details may be made therein without departing from the scope of the invention encompassed by the appended claims.

### REFERENCES

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

For instance, the following references are incorporated by reference:

1. Zammit P S, Relaix F, Nagata Y, Ruiz A P, Collins C A, Partridge T A et al. Pax7 and myogenic progression in skeletal muscle satellite cells. J Cell Sci. 2006; 119(Pt 9):1824-32. doi:10.1242/jcs.02908.

2. Cooper R N, Tajbakhsh S, Mouly V, Cossu G, Buckingham M, Butler-Browne G S. In vivo satellite cell activation via Myf5 and MyoD in regenerating mouse skeletal muscle. J Cell Sci. 1999; 112 (Pt 17):2895-901.

3. Naidu P S, Ludolph D C, To R Q, Hinterberger T J, Konieczny S F. Myogenin and MEF2 function synergistically to activate the MRF4 promoter during myogenesis. Mol Cell Biol. 1995; 15(5):2707-18.

4. Wright W E, Sassoon D A, Lin V K. Myogenin, a factor regulating myogenesis, has a domain homologous to MyoD. Cell. 1989; 56(4):607-17.

5. Yao C C, Ziober B L, Sutherland A E, Mendrick D L, Kramer R H. Laminins promote the locomotion of skeletal myoblasts via the alpha 7 integrin receptor. J Cell Sci. 1996; 109 (Pt 13):3139-50.

6. Johansson S, Svineng G, Wennerberg K, Armulik A, Lohikangas L. Fibronectin-integrin interactions. Front Biosci. 1997; 2:d126-46.

7. Disatnik M H, Rando T A. Integrin-mediated muscle cell spreading. The role of protein kinase c in outside-in and inside-out signaling and evidence of integrin cross-talk. J Biol Chem. 1999; 274(45):32486-92.

8. Bentzinger C F, Wang Y X, von Maltzahn J, Soleimani V D, Yin H, Rudnicki M A. Fibronectin regulates Wnt7a signaling and satellite cell expansion. Cell Stem Cell. 2013; 12(1):75-87. doi:10.1016/j.stem.2012.09.015.

9. Le Grand F, Jones A E, Seale V, Scime A, Rudnicki M A. Wnt7a activates the planar cell polarity pathway to drive the symmetric expansion of satellite stem cells. Cell Stem Cell. 2009; 4(6):535-47. doi:10.1016/j.stem.2009.03.013.

10. Patton B L, Connoll A M, Martin P T, Cunningham J M, Mehta S, Pestronk A et al. Distribution of ten laminin chains in dystrophic and regenerating muscles. Neuromuscul Disord. 1999; 9(6-7):423-33.

11. Tiger C F, Champliaud M F, Pedrosa-Domellof F, Thorned L E, Ekblom P, Gullberg D. Presence of laminin alpha5 chain and lack of laminin alphal chain during human muscle development and in muscular dystrophies. J Biol Chem. 1997; 272(45):28590-5.

12. Talts J F, Andac Z, Gohring W, Brancaccio A, Timpl R. Binding of the G domains of laminin alphal and alpha2 chains and perlecan to heparin, sulfatides, alpha-dystroglycan and several extracellular matrix proteins. EMBO J. 1999; 18(4):863-70. doi:10.1093/emboj/18.4.863.

13. Talts J F, Sasaki T, Miosge N, Gohring W, Mann K, Mayne R et al. Structural and functional analysis of the recombinant G domain of the laminin alpha4 chain and its proteolytic processing in tissues. J Biol Chem. 2000; 275(45):35192-9. doi:10.1074/jbc.M003261200.

14. Hirosaki T, Mizushima H, Tsubota Y, Moriyama K, Miyazaki K. Structural requirement of carboxyl-terminal globular domains of laminin alpha 3 chain for promotion of rapid cell adhesion and migration by laminin-5. J Biol Chem. 2000; 275(29):22495-502. doi:10.1074/jbc.M001326200.

15. Yu H, Talts J F. Beta1 integrin and alpha-dystroglycan binding sites are localized to different laminin-G-domain-like (LG) modules within the laminin alpha5 chain G domain. Biochem J. 2003; 371(Pt 2):289-99. doi:10.1042/BJ20021500.

16. Domogatskaya A, Rodin S, Tryggvason K. Functional diversity of laminins. Annu Rev Cell Dev Biol. 2012; 28:523-53. doi:10.1146/annurev-cellbio-101011-155750.

17. Motohashi N, Asakura Y, Asakura A. Isolation, culture, and transplantation of muscle satellite cells. J Vis Exp. 2014(86). doi:10.3791/50846.

18. Lagord C, Soulet L, Bonavaud S, Bassaglia Y, Rey C, Barlovatz-Meimon G et al. Differential myogenicity of satellite cells isolated from extensor digitorum longus (EDL) and soleus rat muscles revealed in vitro. Cell Tissue Res. 1998; 291(3):455-68.

19. Shefer G, Wleklinski-Lee M, Yablonka-Reuveni Z. Skeletal muscle satellite cells can spontaneously enter an alternative mesenchymal pathway. J Cell Sci. 2004; 117(Pt 22):5393-404. doi:10.1242/jcs.01419.

20. Podleski T R, Greenberg I, Schlessinger J, Yamada K M. Fibronectin delays the fusion of L6 myoblasts. Exp Cell Res. 1979; 122(2):317-26.

21. Wilschut K J, Haagsman H P, Roelen B A. Extracellular matrix components direct porcine muscle stem cell behavior. Exp Cell Res. 2010; 316(3):341-52. doi:10.1016/j.yexcr.2009.10.014.

22. Mitchell P O, Mills T, O'Connor R S, Kline E R, Graubert T, Dzierzak E et al. Sca-1 negatively regulates proliferation and differentiation of muscle cells. Dev Biol. 2005; 283(1):240-52. doi:10.1016/j.ydbio.2005.04.016.

23. Przewozniak M, Czaplicka I, Czerwinska A M, Markowska-Zagrajek A, Moraczewski J, Streminska W et al. Adhesion proteins—an impact on skeletal myoblast differentiation. PLoS One. 2013; 8(5):e61760. doi: 10.1371/journal.pone.0061760.

24. Brzoska E, Bello V, Darribere T, Moraczewski J. Integrin alpha3 subunit participates in myoblast adhesion and fusion in vitro. Differentiation. 2006; 74(2-3):105-18. doi: 10.1111/j .1432-0436.2005.00059.x.

25. Danen E H, Sonneveld P, Brakebusch C, Fassler R, Sonnenberg A. The fibronectin-binding integrins alpha5beta1 and alphavbeta3 differentially modulate RhoA-GTP loading, organization of cell matrix adhesions, and fibronectin fibrillogenesis. J Cell Biol. 2002; 159(6):1071-86. doi : 10.1083/j cb.200205014.

26. Sechler J L, Cumiskey A M, Gazzola D M, Schwarzbauer J E. A novel RGD-independent fibronectin assembly pathway initiated by alpha4beta1 integrin binding to the alternatively spliced V region. J Cell Sci. 2000; 113 (Pt 8):1491-8.

27. Urciuolo A, Quarta M, Morbidoni V, Gattazzo F, Molon S, Grumati P et al. Collagen VI regulates satellite cell self-renewal and muscle regeneration. Nat Commun. 2013; 4:1964. doi:10.1038/ncomms2964.

28. Trensz F, Lucien F, Couture V, Sollrald T, Drouin G, Rouleau A J et al. Increased microenvironment stiffness in damaged myofibers promotes myogenic progenitor cell proliferation. Skelet Muscle. 2015; 5:5. doi:10.1186/s13395-015-0030-1.

29. Lacraz G, Rouleau A, Couture V, Sollrald T, Drouin G, Veillette N et al. Increased Stiffness in Aged Skeletal Muscle Impairs Muscle Progenitor Cell Proliferative Activity. PLoS One. 2015; 10(8):e0136217. doi:10.1371/journal.pone.0136217.

30. Engler A J, Sen S, Sweeney H L, Discher D E. Matrix elasticity directs stem cell lineage specification. Cell. 2006; 126(4): 677-89. doi: 10.1016/j.cell.2006.06.044.

31. Engler A J, Griffin M A, Sen S, Bonnemann C G, Sweeney H L, Discher D E. Myotubes differentiate optimally on substrates with tissue-like stiffness: pathological implications for soft or stiff microenvironments. J Cell Biol. 2004; 166(6):877-87. doi:10.1083/jcb.200405004.

32. Rossi C A, Flaibani M, Blaauw B, Pozzobon M, Figallo E, Reggiani C et al. In vivo tissue engineering of functional skeletal muscle by freshly isolated satellite cells embedded in a photopolymerizable hydrogel. FASEB J. 2011; 25(7):2296-304. doi:10.1096/fj.10-174755.

33. Hughes C S, Postovit L M, Lajoie G A. Matrigel: a complex protein mixture required for optimal growth of cell culture. Proteomics. 2010; 10(9):1886-90. doi:10.1002/pmic.200900758.

34. Ringelmann B, Roder C, Hallmann R, Maley M, Davies M, Grounds M et al. Expression of laminin alpha1, alpha2, alpha4, and alpha5 chains, fibronectin, and tenascin-C in skeletal muscle of dystrophic 129ReJ dy/dy mice. Exp Cell Res. 1999; 246(1):165-82. doi:10.1006/excr.1998.4244.

35. Tiger C F, Gullberg D. Abscence of laminin alphal chain in the skeletal muscle of dystrophic dy/dy mice. Muscle Nerve. 1997; 20(12):1515-24.

36. Goudenege S, Lamarre Y, Dumont N, Rousseau J, Frenette J, Skuk D et al. Laminin-111: a potential therapeutic agent for Duchenne muscular dystrophy. Mol Ther. 2010; 18(12):2155-63. doi:10.1038/mt.2010.165.

37. Gawlik K I, Oliveira B M, Durbeej M. Transgenic expression of Laminin alphal chain does not prevent muscle disease in the mdx mouse model for Duchenne muscular dystrophy. Am J Pathol. 2011; 178(4):1728-37. doi:10.1016/j.ajpath.2010.12.030.

38. Law P K, Goodwin T G, Fang Q, Deering M B, Duggirala V, Larkin C et al. Cell transplantation as an experimental treatment for Duchenne muscular dystrophy. Cell Transplant. 1993; 2(6):485-505.

39. Law P K, Goodwin T G, Fang Q, Duggirala V, Larkin C, Florendo J A et al. Feasibility, safety, and efficacy of myoblast transfer therapy on Duchenne muscular dystrophy boys. Cell Transplant. 1992; 1(2-3):235-44.

40. Law P K, Goodwin T G, Fang Q W, Chen M, Li H J, Florendo JA et al. Myoblast transfer therapy for Duchenne muscular dystrophy. Acta Paediatr Jpn. 1991; 33(2):206-15.

41. Mendell J R, Kissel J T, Amato A A, King W, Signore L, Prior T W et al. Myoblast transfer in the treatment of Duchenne's muscular dystrophy. N Engl J Med. 1995; 333(13):832-8. doi:10.1056/NEJM199509283331303.

42. Montarras D, Morgan J, Collins C, Relaix F, Zaffran S, Cumano A et al. Direct isolation of satellite cells for skeletal muscle regeneration. Science. 2005; 309(5743):2064-7. doi:10.1126/science.1114758.

43. Grefte S, Vullinghs S, Kuijpers-Jagtman A M, Torensma R, Von den Hoff J W. Matrigel, but not collagen I, maintains the differentiation capacity of muscle derived cells in vitro. Biomed Mater. 2012; 7(5):055004. doi:10.1088/1748-6041/7/5/055004.

44. Kanisicak O, Mendez J J, Yamamoto S, Yamamoto M, Goldhamer D J. Progenitors of skeletal muscle satellite cells express the muscle determination gene, MyoD. Dev Biol. 2009; 332(1): 131-41. doi: 10.1016/j.ydbio.2009.05.554.

45. Wilschut K J, van Tol H T, Arkesteijn G J, Haagsman H P, Roelen B A. Alpha 6 integrin is important for myogenic stem cell differentiation. Stem Cell Res. 2011; 7(2):112-23. doi:10.1016/j.scr.2011.05.001.

46. Jarocha D, Stangel-Wojcikiewicz K, Basta A, Majka M. Efficient myoblast expansion for regenerative medicine use. Int J Mol Med. 2014; 34(1):83-91. doi:10.3892/ijmm.2014.1763.

47. Salani S, Donadoni C, Rizzo F, Bresolin N, Comi G P, Corti S. Generation of skeletal muscle cells from embryonic and induced pluripotent stem cells as an in vitro model and for therapy of muscular dystrophies. J Cell Mol Med. 2012; 16(7):1353-64. doi:10.1111/j.1582-4934.2011.01498.x.

48. Hosoyama T, McGivern J V, Van Dyke J M, Ebert A D, Suzuki M. Derivation of myogenic progenitors directly from human pluripotent stem cells using a sphere-based culture. Stem Cells Transl Med. 2014; 3(5):564-74. doi:10.5966/sctm.2013-0143.

49. Awaya T, Kato T, Mizuno Y, Chang H, Niwa A, Umeda K et al. Selective development of myogenic mesenchymal cells from human embryonic and induced pluripotent stem cells. PLoS One. 2012; 7(12):e51638. doi:10.1371/journal.pone.0051638.

50. Maffioletti S M, Gerli M F, Ragazzi M, Dastidar S, Benedetti S, Loperfido M et al. Efficient derivation and inducible differentiation of expandable skeletal myogenic cells from human ES and patient-specific iPS cells. Nat Protoc. 2015; 10(7):941-58. doi:10.1038/nprot.2015.057.

51. Spenle C, Simon-Assmann P, Orend G, Miner J H. Laminin α5 guides tissue patterning and organogenesis. Cell Adh. Migr. 2013; 7(1):90-100. doi:10.4161/cam.22236.

52. Macdonald P R, Lustig A, Steinmetz M O, Kammerer R A. Laminin chain assembly is regulated by specific coiled-coil interactions. J Struct. Biol. 2010; 170(2):398-405. doi: 10.1016/j.jsb.2010.02.004.

53. Siler U, Rousselle P, Müller C A, Klein G. Laminin γ2 chain as stromal cell marker of the human bone marrow microenvironment. Br. J. Haematol. 2002; 119:212-220.

What is claimed is:

1. A method for producing a muscle progenitor cell comprising culturing a muscle progenitor cell in the presence of one or more laminin α5 proteins, wherein the cultured progenitor cell has improved differentiation and engraftment potential as compared to cells not cultured in laminin α5.

2. The method of claim 1, wherein the laminin α5 is one or more of laminin 521, laminin 511, laminin 522, or laminin 523.

3. The method of claim 2, wherein the laminin α5 is laminin 521.

4. The method of claim 1, wherein the laminin α5 is recombinant.

5. The method of claim 1, wherein the laminin α5 is human.

6. The method of any one of the preceding claims, wherein the progenitor cell is passaged more than 5 times.

7. The method of any one of the preceding claims, wherein the progenitor cell is passaged more than 10, 15, 20 or 25 times.

8. The method of any one of the preceding claims, wherein the progenitor cell is expanded in culture by 9000-100000-fold.

9. The method of any one of the preceding claims, wherein the cultured progenitor cell has improved fusion capacity.

10. The method of any one of the preceding claims, wherein the cultured progenitor cell has diminished spontaneous differentiation.

11. The method of any one of the preceding claims, wherein the method further comprises culturing the progenitor cell in the presence of one or more additional extracellular matrix (ECM) agents.

12. The method of any one of the preceding claims, wherein the method further comprises transferring the cultured progenitor cell to another substrate comprising one or more additional ECM agents.

13. The method of claim 11 or 12, wherein the one or more additional ECM agents comprise one or more of laminin other than laminin α5, fibronectin, gelatin, collagen, hydrogel, and matrigel.

14. The method of any one of claim 12 or 13, wherein the cells not cultured in laminin α5 are cultured in the presence of laminin 211, laminin 111, fibronectin, gelatin, collagen, hydrogel, or matrigel.

15. A method for treating or preventing a neuromuscular disease or disorder, comprising:
preparing a muscle progenitor cell as in any one of claims 1-14 and administering an effective amount of the cultured progenitor cell to a subject in need thereof.

16. The method of claim 15, wherein the neuromuscular disease or disorder is a muscular dystrophy.

17. The method of claim 16, wherein the muscular dystrophy is one or more of Becker muscular dystrophy, congenital muscular dystrophy, Duchenne muscular dystrophy, distal muscular dystrophy, Emery-Dreifuss muscular dystrophy, facioscapulohumeral muscular dystrophy, limb-girdle muscular dystrophy, myotonic muscular dystrophy, and oculopharyngeal muscular dystrophy.

18. The method of any one of claims 15-17, further comprising administration of an additional therapeutic agent.

19. A cell culture substrate comprising laminin α5 and a muscle progenitor cell.

20. The cell culture substrate of claim 19, wherein the substrate comprises a tissue culture dish or flask.
